(19) United States
(12) Patent Application Publication
(10) Pub. No.: US 2004/0220276 A1
(43) Pub. Date: Nov. 4, 2004

(54) Coated granules of allylamine-or benzylamine-anti-mycotics

(76) Inventors: Gerard Cousin, Villemeux/Eure (FR); Etienne Bruna, Jouy (FR); Edouard Gendrot, Garnay (FR)

Correspondence Address: CAESAR, RIVISE, BERNSTEIN,, COHEN & POKOTILOW, LTD., 11TH FLOOR, SEVEN PENN CENTER, PHILADELPHIA, PA, 19103-2212, US

(21) Appl. No.: 10/484,588

(22) PCT Filed: Jul. 19, 2002
(86) PCT No.: PCT/EP02/08103

(30) Foreign Application Priority Data
Jul. 26, 2001 (EP) 01402027.5

(51) Int. Cl. A61K 31/137; A61K 9/14

(52) U.S. Cl. 514/649; 424/489

(57) ABSTRACT

Granules of allylamine- or benzylamine-anti-mycotic or one of its pharmaceutically acceptable salts, characterized in that the granules are coated and that they contain:—microcrystals of an allylamine- or benzylamine-anti-mycotic or one of its pharmaceutically acceptable salts, —at least one binder selected from the group consisting of cellulosic polymers, such as ethylcellulose, hydroxypropylcellulose and hydroxypropylmethylcellulose, acrylic polymers, povidones, polyvinyl alcohols and mixtures thereof—optionally a diluent agent selected from the group comprising cellulosic derivatives, starches, lactose, polyols such as mannitol and/or an antistatic agent selected from the group comprising micronised or non micronised talc and/or a permeabilising agent selected from the group comprising colloidal silica and precipitated silica. Process for obtaining such granules and orodispersible tablets comprising them. Use of said orodispersible tablets.

The present invention concerns coated granules of an allylamine- or benzylamine-anti-mycotic, a process for the preparation thereof and orodispersible tablets comprising coated granules of an allylamine or benzylamine-anti-mycotic.

In the context of the present invention, the term “orodispersible tablets” means tablets which are able to disintegrate in the buccal cavity in less than 60 seconds, preferably in less than 40 seconds, upon contact with saliva by formation of an easy-to-swallow suspension.

Butenafine, naftifine and terbinafine are well known synthetic anti-mycotics. Terbinafine and naftinine are examples of allylamine-anti-mycotics and butenaftine is an example of benzylamine-anti-mycotic.

Terbinafine is known from e.g. EP-A-24587. It is acknowledged in the art and is commercially available, in particular as a swallowable tablet, under the trade name Lamisil®.

Allylamine- and benzylamine-anti-mycotics are highly active via both topical and oral administration. While numerous pharmaceutical compositions for topical and oral administration have been proposed, there still exists a need for commercially acceptable allylamine or benzylamine-anti-mycotic formulations for oral administration with good patient convenience and acceptance, especially for children or the elderly.

One particular difficulty in the formulation of allylamine- or benzylamine-anti-mycotics in oral pharmaceutical compositions is their unpleasant, bitter, taste and low physical integrity.

Furthermore, some patients, especially children and the elderly, experience difficulties swallowing the tablets, even with liquids.

It is estimated that 50% of the population has problems swallowing the tablets. Said problem leads to poor or even noncompliance with the treatment and thus has a negative impact on the efficiency of the treatment (H. Seager, 1998, J. Pharm. Pharmacol. 50, 375-382).

Oral disintegrable multiparticulate tablets have already been described in EP 548356, EP1003484, WO 00/27357 and WO00/51568, the content of which is hereby incorporated by reference. The active ingredient is in the form of coated microcrystals or coated microgranules.

Up to now, no oral formulations of allylamine- or benzylamine-anti-mycotic exist which are specifically suitable for patients having difficulties when swallowing or for patients taking the drugs with no liquids.

It is thus highly desirable to remedy this situation and to develop an orodispersible tablet containing an allylamine- or benzylamine-anti-mycotic, which has taste-masking properties and presents a pleasant palatability such that the administration of the tablet is not unpleasant for the patient.

The Applicant has now surprisingly found that these characteristics can be obtained by formulating a tablet containing an allylamine- or benzylamine-anti-mycotic as active principle in the form of coated granules, and a mixture of excipients containing at least one disintegration agent, a soluble agent and a lubricant, and optionally a swelling agent, a permeabilising agent, sweeteners and flavouring agents.

Such a tablet allows the obtaining of pharmacokinetical parameters at least equivalent to those which are obtained with conventional oral formulations of allylamine- or benzylamine-anti-mycotic, especially terbinafine.

Surprisingly, although the tablets according to the invention disintegrate in the buccal cavity and present a liberation of the active principle which is equivalent to the conventional formulation, they nevertheless have a pleasant taste.

Furthermore, these rapidly disintegrating compositions of the invention were found to show high stability and physical integrity, e.g. during storage, handling, packaging and the like, without limiting the disintegration performance of the composition.

The tablets according to the invention comprise coated granules of an allylamine- or benzylamine- anti-mycotic, or one of its pharmaceutically acceptable salts.

The allylamine- or benzylamine-anti-mycotics, such as butenafine, naftifine and terbinafine may be used e.g. in free base form or in acid addition salt form. An acid addition salt form may be prepared from the free base form in a conventional manner and vice-versa. Examples of suitable acid addition salt forms include hydrochloride, lactate and ascorbate, preferably hydrochloride.

The present invention also relates to said coated granules.

The taste-masking of the allylamine- or benzylamine-anti-mycotic is achieved by coating, granulated microcrystals of the allylamine- or benzylamine-anti-mycotic with one or more polymers.

According to an advantageous embodiment of the invention, the granules of the allylamine- or benzylamine-anti-mycotic, or one of its pharmaceutically acceptable salts, are characterized in that the granules are coated and that they contain:

microcrystals of the allylamine- or benzylamine-anti-mycotic, or one of its pharmaceutically acceptable salts,

at least one binder,

optionally a diluent agent and/or an antistatic agent, and/or a permeabilising agent.

The binder is selected from the group consisting of cellulosic polymers, acrylic polymers, povidones, polyvinylalcohols and mixtures thereof, preferably polyvinylpyrrolidone (Kollidon K30).

Optionally, in order to enhance the granulation of the allylamine- or benzylamine-anti-mycotic or one of its pharmaceutically acceptable salts, a diluent agent is used.

The diluent agent is selected from the group comprising cellulosic derivatives and is preferably selected from the group consisting of microcrystalline cellulose, starches, lactose and polyols such as mannitol.

In order to facilitate the fluidification of the composition during the granulation step, it is possible to use a permeabilising agent, and/or an antistatic agent.

Said permeabilising agent is selected from the group comprising colloidal silica (Aerosil®) precipitated silica (Syloïd® FP244).

Said antistatic agent is selected from the group of micronised or non micronised talc.

According to another embodiment of the invention, the granules comprise:

from 10% to 80% of granules of an allylamine- or benzylamine-anti-mycotic, or one of the pharmaceutically acceptable salts thereof,

at most 15%, preferably at most 5% by weight of the binder, relative to the weight of the allylamine- or benzylamine-anti-mycotic, or one of the pharmaceutically acceptable salts thereof,

at most 2%, preferably 0.5% by weight of the antistatic agent, relative to the weight of the granules of allylamine- or benzylamine-anti-mycotic, or one of the pharmaceutically acceptable salts thereof.

In order to ensure efficient taste masking, and a dissolution profile of the active substance such that more than 80% of the active substance is released in 30 minutes, preferably 70% is released in 15 minutes, the granules are coated with a coating composition containing at least one coating polymer selected from the group consisting of cellulosic polymers, acrylic polymers and their mixtures.

Among the cellulosic polymers, ethylcellulose, hydroxypropylcellulose (HPC) and hydroxypropylmethylcellulose (HPMC), are advantageously used.

Among the acrylic polymers, insoluble acrylate ammonio-methacrylate copolymer (Eudragit®RL100 or RS100 or Eudragit® RL30D or RS30D), polyacrylate (Eudragit® NE), or methacrylic copolymers (Eudragit® L100-55 or Eudragit® L30D), are advantageously used, alone, in combination or in admixture with pH-dependant polymers. A mixture of Eudragit® RL100 with Eudragit®L100-55 is preferred,. with a ratio comprised respectively of between 70/30 and 30/70, more preferably 30/70.

The prepared coating liquid is either water-based or prepared with organic solvents. According to an advantageous embodiment, this coating liquid is suitable to be sprayed with conventional spray layering equipment.

Optionally a permeabilising agent, plasticizers, and soluble agents are added as coating additives.

The plasticizer is selected in the group consisting of triacetine, triethylacetate, triethylcitrate (Eudraflex®), ethyl phthalate, or mixtures thereof. The plasticizer is used in proportions of at most about 30%, preferably 10% by weight of the coating polymers.

The soluble agents are selected in particular among the polyols.

Advantageously the coated granules according to the invention present a granulometric repartition such that at least 50%, preferably at least 70% of the granules have a particle size ranging between 125 and 355 μm and less than 15% of the granules have a particle size less than 90 μm.

The particle sizes are measured according to conventional methods, preferably by sieving.

In a particular embodiment, the coated granules according to the invention comprise:

from 10% to 95% of granules of the allylamine- or benzylamine-anti-mycotic, or one of its pharmaceutically acceptable salts, preferably terbinafine HCl,

from 5 to 60%, preferably 5 to 40% and even more preferably from 10 to 20% of a coating polymer, preferably a mixture of Eudragit RL100/Eudragit L100-55, the percentages being expressed by weight relative to the weight of the granules of the allylamine- or benzylamine-anti-mycotic, or one of its pharmaceutically acceptable salts,

from 0 to 30%, preferably 10% by weight of plasticizers, preferably ethyl phthalate, the percentages being expressed by weight relative to the weight of the coating polymer,

from 0 to 10% of a permeabilising agent, preferably colloidal silica, the percentages being expressed by weight relative to the weight of the coating polymer, and

from 0 to 10% of an antistatic agent, preferably micronised talc, the percentages being expressed by weight relative to the weight of the coating polymer.

According to another embodiment of the invention, the coated granules of the allylamine or benzylamine-anti-mycotic, or one of its pharmaceutically acceptable salts, further comprise an overcoating which consists essentially in acrylic polymer, cellulosic polymer, thermoplastic polymer or mixtures thereof.

Optionally the overcoating ratio is from about 10 to about 60% by weight relative to the coated granules.

The invention also concerns a process for preparing coated granules of the allylamine- or benzylamine-anti-mycotic, or one of its pharmaceutically acceptable salts;

The process comprises the successive steps consisting in:

dry mixing the microcrystals of the allylamine- or benzylamine-anti-mycotic, or one of its pharmaceutically acceptable salts optionally with an antistatic agent and/or a diluent agent, and/or a permeabilising agent;

granulating the thus obtained mixture by spraying thereon a solution or a suspension of the binder,

coating the thus obtained granules by spraying thereon a suspension, a dispersion or a solution of the coating composition,

optionally, applying an overcoating based on acrylic polymer, cellulosic polymer, thermoplastic polymer or mixtures thereof,

drying the thus obtained coated granules.

In this process the mixing, granulating and coating steps can be performed in different apparatuses or in the same apparatus, each step being performed in the presence of a mixture of excipients which are identical or different.

In an advantageous embodiment, each step is performed on a fluidized air-bed, such as for example, but not limited to Glaft GPCG-1, GPCG-5 or GPCG 120.

For granulating, bottom spray granulation, tangential spray granulation, top spray granulation or high shear granulation can be used, the bottom spray granulation being preferred.

For coating, bottm, top and tangential spray methods can be used as well as layering method, bottom spray method of coating being preferred.

The optional overcoating step can be performed using the same method as used for the coating step.

The coated granules thus obtained are particularly useful for incorporation in the manufacture of rapidly disintegratable tablets, which are also another object of the present invention.

The present invention also relates to orodispersible tablets, i.e. rapidly disintegratable tablets which are able to disintegrate in the buccal cavity upon contact with saliva by formation of an easy-to-swallow suspension, in less than 60 seconds, preferably in less than 40 seconds. Said tablets are characterized by the fact that they contain the above described coated granules, and a mixture of excipients comprising at least one disintegrating agent, a soluble diluent agent, a lubricant and optionally a swelling agent, a permeabilising agent, sweeteners, flavoring agents and colors.

The disintegration time corresponds to the time between the moment when the tablet is placed in the buccal cavity in contact with saliva and the moment when the suspension resulting from the disintegration without chewing of the tablet is swallowed.

According to an advantageous embodiment, the tablet according to the invention, have a hardness of not less than 15 N, when measured with the test method of the European Pharmacopeia (2.9.8) According to an advantageous embodiment, the tablet according to the invention, contains coated granules of an allylamine- or benzylamine-anti-mycotic, or one of its pharmaceutically acceptable salts, and a mixture of excipients, the ratio of the mixture of excipients to the coated granules is 0.4 to 10, preferably 0.4 to 5 and even more preferably 1 to 2 parts by weight, the mixture of excipients comprising:

at least one disintegrating agent,

a soluble diluent agent which presents binding properties,

a lubricant,

a permeabilising agent,

and optionally sweeteners, flavorings and colors.

The disintegrating agents are selected from the group consisting of croscarmellose, available as e.g. Ac-di-sol®, crospovidone available as e.g. Kollidon CL®, and mixtures thereof.

The soluble diluent agent with binding properties consists of a polyol having less than 13 carbon atoms and being either in the form of the directly compressible product with an average particle size of 100 to 500 μm, or in the form of a powder with an average particle size of less than 100 μm, this polyol preferably being selected from the group comprising mannitol, xylitol, sorbitol and maltitol, it being understood that sorbitol cannot be used alone and that, in the case where there is only one soluble diluent agent with binding properties, it is used in the form of the directly compressible product, whereas in the case where there are at least two soluble diluent agents with binding properties, one is present in the directly compressible form and the other is present in powder form, it then being possible for the polyols to be the same, the ratio of directly compressible polyol to powder polyol being 99/1 to 20/80, preferably 80/20 to 20/80.

The proportion of disintegrating agents being 1 to 20% by weight, preferably 5 to 15% by weight, in the case of a mixture, each disintegrating agent being comprised between 0.5 and 15% by weight, preferably 3 to 8% by weight, and the proportion of soluble agent being 30 to 90% by weight, preferably 30 to 50% by weight, based in each case on the weight of the tablet.

The lubricant is selected from the group consisting of magnesium stearate, stearic acid, sodium stearyl fumarate, micronised polyoxyethyleneglycol (micronised Macrogol 6000), leukine, sodium benzoate and mixtures thereof.

The amount of lubricant is from 0.02 to 2 percent, preferably from 0.5 to 1 percent (weight of the lubricant /total weight of the tablet).

The lubricant can be dispersed within the mixture of excipients, or according to an advantageous embodiment, it can be dispersed on the surface of the tablet.

The permeabilizing agent used is a compound selected from the group comprising especially silicas with a high affinity for aqueous solvents, such as the precipitated silica better known by the trademark Syloïd® maltodextrins, β-cyclodextrins and mixtures thereof.

The permeabilizing agent allows the creation of a hydrophilic network which facilitates the penetration of saliva and hence assists the disintegration of the tablet.

The proportion of permeabilising agent is between 0.5 and 5% by weight, based on the weight of the tablet.

A sweetener and optionally a flavoring and a color are also included in the mixture of excipients forming part of the composition of the tablets according to the invention.

The sweetener can be selected from the group comprising especially aspartame, potassium acesulfame, sodium saccharinate, neohesperidin dihydrochalcone, sucralose, monoammonium glycyrrhizinate, and mixtures thereof.

The flavorings and colors are those conventionally used in pharmacy for the preparation of tablets.

Determination of workable proportions in any particular instance will generally be within the capability of the man skilled in the art. All indicated proportions and relative weight ranges described above are accordingly to be understood as being indicative of preferred or individually inventive teachings only and not as limiting the invention in its broadest aspect.

Details concerning any of the excipients of the invention may be found in Fiedler, H. P. “Lexikon der Hilfsstoffe für Pharmazie, Kosmetik und angrenzende Gebiete”, Editio Cantor Verlag Aulendorf, Aulendorf, 4th revised and expanded edition (1996); “Handbook of Pharmaceutical Excipients”, 2nd Edition, Editors A. Wade and P. J. Weller (1994), Joint publication of American Pharmaceutical Association, Washington, USA and The Pharmaceutical Press, London, England; or may be obtained from the relevant manufacturers, the contents of which are hereby incorporated by reference.

For compressing the mixture of coated granules and excipients into a tablet, various punches may be used, with diameters comprised between 10 and 14 mm, depending upon the dosage of the tablet.

Various shapes may be used, such as for example, flat shape, advantageously with bevelled edges or polo punches.

The orodispersible tablets of the present invention show fast disintegration in the buccal cavity upon contact with saliva without chewing, in less than 60 seconds, preferably in less than 40 seconds, have a pleasant taste and palatability and thus have particularly good patient convenience and patient acceptance due to their increased ease of administration and ingestion.

In addition the tablets of the invention show surprisingly high physical stability and are easy to handle and package.

The tablets of this invention are useful for the known indications of the allylamine- or benzylamine-anti-mycotics, e.g. for the following conditions: onychomycosis caused by dermatophyte fungi, tinea capitis, fungal infections of the skin, for the treatment of tinea corporis, tinea cruris, tinea pedis, and yeast infections of the skin caused by the genus Candida, e.g. Candida albicans, systemic mycosis, mycosis by azole-resistant strains, e.g. in combination with a 14-alpha-methyldimethylase inhibitor, or infections with Helicobacter pylori.

The tablets are particularly effective in treating tinea capitis in e.g. children.

The utility of the tablets of the present invention may be observed in standard bioavailability tests or standard animal models, for example ascertaining dosages of the present tablets giving blood levels of the allylamine- or benzylamine-anti-mycotic, expressed as allylamine- or benzylamine-anti-mycotic base equivalent to blood levels giving a therapeutical effect on administration of known allylamine- or benzylamine-anti-mycotic oral dosage forms, e.g. a tablet. typical doses are in the range of 1 mg/kg to 10 mg/kg, e.g. 1.5 mg/kg to 5 mg/kg, or e.g. 3 to 4 mg/kg body weight of allylamine- or benzylamine-anti-mycotic per day.

The appropriate dosage will, of course, vary depending upon, for example, the host and the nature and severity of the condition being treated. However, in general satisfactory results in animals are indicated to be obtained at daily treatments with doses from about 1 mg/kg to about 10 mg/kg animal body weight. In humans an indicated daily dosage is in the range from about 10 mg to about 1000 mg per day, conveniently administered, for example, in divided doses up to four times a day or once daily. Preferred dosages, expressed as allylamine- or benzylamine-anti-mycotic base, for children weighing less than 20 kg may be about 62.5 mg once daily, for children weighing 20 to 40 kg about 125 mg once daily, for children weighing more than 40 kg about 250 mg once daily, and for adults from about 250 mg to about 500 mg once daily.

The invention is illustrated more in detail in the following examples.

### EXAMPLES

In the examples below, the following products are used:

HPMC: hydroxypropylmethylcellulose

methacrylic copolymers (Eudragit®L30D-55, Eudragit® E100, Eudragit® L100D-55, sold by Röhm),

Kollidon 30: polyvinylpyrrolidone sold by BASF,

Aquacoat: ethylcellulose sold by FMC,

Ac-Di-Sol: sodium croscarmellose, sold by FMC,

Kollidon CL: crospovidone, sold by BASF,

Eudraflex: triethylcitrate, sold by Röhm,

Syloïd 244 FP: precipitated silica.

### Example 1: [9831/05]

Granulating Step

400 g of terbinafine HCl were granulated in a fluidized bed GPCG1 GLATT with 1432 g of an aqueous suspension including 80 g of HPMC and 400 g of terbinafine HCl, by spraying onto the powder.

Coating Step

The thus obtained granules were coated by spraying thereon 12986 g of an aqueous suspension containing 400 g of Eudragit® L30D-55 and 120 g of Eudragit® E100, at a pH of not less than 2.5.

The amount of coating was of 20% by weight with respect to the weight of the granules of terbinafine HCl for example 1A and of 30% by weight with respect to the weight of the granules of terbinafine HCl for example 1B.

The dissolution rates of said granules were measured as follows:

An amount of granules corresponding to 125 mg of terbinafine base was added once to 500 ml of the European pharmacopoeia buffer of pH=3, under stirring at 50 rpm. A sample of the solution was analyzed by HPLC at 280 nm, at t=2, 5, 10, 15 and 30 minutes, in order to determine the percentage of dissolved terbinafine.

The results are given in the following table 1.:

TABLE 1

| | Coated granules | Coated granules |
|---|---|---|
| | of example 1A | of example 1B |
| 2 minutes | 41.8% | 23.3% |
| 5 minutes | 75.8% | 62.7% |
| 10 minutes | 90.8% | 84.8% |
| 15 minutes | 95.4% | 92.1% |
| 30 minutes | 96.8% | 95.3% |

After 30 minutes, more than 80% of terbinafine was dissolved, however the taste was not very pleasant, due to a non compatibility between Eudragit® E100 and terbinafine HCl.

### Example 2: [9831/06]

Granulating Step

800 g of terbinafine HCl were granulated in a fluidized bed GPCG1 with 720 g of ethanol in which 80 g of polyvinylpyrrolidone was dissolved.

Coating Step:

The thus obtained granules were coated by spraying thereon an homogeneous mixture of 720 g of HPMC in water (10% w/w) and 593,6 g of a mixture of Aquacoat ECD30 and triethylcitrate (20% by weight of polymer).

The amount of coating was of 20% by weight with respect to the weight of the granules of terbinafine HCl for example 2A and of 30% by weight with respect to the weight of the granules of terbinafine HCl for example 2B.

The dissolution rates of said granules were measured as indicated in example 1 above.

The results are given in the following table 2.:

TABLE 2

| | Coated granules | Coated granules |
|---|---|---|
| | of example 2A | of example 2B |
| 2 minutes | 25.8% | 25.7% |
| 5 minutes | 54.9% | 53.8% |
| 10 minutes | 81.6% | 82.3% |
| 15 minutes | 91.5% | 92.3% |
| 30 minutes | 97.6% | 99.3% |

More than 80% of terbinafine is dissolved after 30 minutes, taste-masking is efficient.

### Example 3: [9831/07]

Granulating Step:

880 g granules of terbinafine HCl were prepared as described in example 1 (granulating step).

Coating Step:

The thus obtained granules were coated by spraying thereon 1840 g of homogeneous dispersion containing 720 g of HPMC in water (10% w/w) and 1120 g of Eudragit® L30D .

The amount of coating was of 20% by weight with respect to the weight of the granules of terbinafine HCl for example 3A and of 30% by weight with respect to the weight of the granules of terbinafine HCl for example 3B.

The dissolution rates of said granules were measured as indicated in example 1 above.

The results are given in the following table 3:

TABLE 3

| | Coated granules | Coated granules |
|---|---|---|
| | of example 3A | of example 3B |
| 2 minutes | 24.2% | 18.8% |
| 5 minutes | 59.1% | 47.6% |
| 10 minutes | 78.5% | 76.7% |
| 15 minutes | 88.6% | 89.2% |
| 30 minutes | 94.9% | 97.8% |

More than 80% of terbinafine is dissolved after 30 minutes.

### Example 4: [9831/09]

500 g of mannitol were introduced in a fluidized bed GPCG-1

1000 g of terbinafine HCl were suspended with 1000 g of ethanol and 2000 g of acetone

300 g of PVP K90 were then dissolved in the suspension. The obtained suspension was sprayed onto the mannitol cores.

Coating Step

The thus obtained granules were coated by spraying thereon a mixture of the same composition as for example 3 (granulating step).

The amount of coating was of 30% by weight with respect to the weight of the granules of terbinafine HCl.

The dissolution rate of said granules was measured as indicated in example 1 above.

The results are given in the following table 4.:

TABLE 4

| | Coated granules of example 4 |
|---|---|
| 2 minutes | 30.2% |
| 5 minutes | 56.9% |
| 10 minutes | 81.2% |
| 15 minutes | 89.9% |
| 30 minutes | 93.0% |

More than 80% of terbinafine HCl is dissolved after 30 minutes.

### Example 5: [9831/10]

Granulation Step

Granulation of 800 g of terbinafine HCl was prepared as in example 1 (granulating step)

Coating Step

The thus obtained granules were coated by spraying thereon 1392 g of an aqueous dispersion of Eudragit® L30D and Eudragit®RL30D, respectively in a ratio of (70/30), with triacetine as plasticizer.

The amount of coating was of 30% by weight with respect to the weight of the granules of terbinafine HCl.

The dissolution rates of said granules were measured as indicated in example 1 above.

The results are given in the following table 5.

TABLE 5

| | Coated granules of example 5 |
|---|---|
| 2 minutes | 19.1 |
| 5 minutes | 41.4 |
| 10 minutes | 63.3 |
| 15 minutes | 75.5 |
| 30 minutes | 89.2 |

More than 80% of terbinafine is dissolved after 30 minutes, taste-masking is effective.

### Example 6: [99026/0057]

Tablets according to the invention were prepared as follows.

Granulation Step:

TABLE 6a

| Components | Quantity (g) | % |
|---|---|---|
| terbinafine HCl | 8000 | 94.79 |
| Kollidon K30 | 400 | 4.74 |
| Syloïd 244 FP | 40 | 0.47 |
| Ethanol USP/PE | 3600 | |

The components given in Table 6a above were introduced in a fluidized bed. During the granulation the temperature was maintained between 19 to 22° C.

Coating Step:

The obtained granules were then coated with the coating composition given in table 6b below:

TABLE 6b

| Components | Amount (g) | % |
|---|---|---|
| Granules of terbinafine | 8000 | 65.27 |
| HCl | | |
| Eudragit ® L100-55 | 2128 | 17.36 |
| Eudragit ® RL100 | 912 | 7.44 |
| Ethyl phthalate | 304 | 2.48 |
| Syloïd 244 FP | 912 | 7.44 |
| Ethanol USP/PE | 3600 | |

The coating ratio was 38% by weight with respect to the weight of the granules of terbinafine HCl.

Said coated granules present a good dissolution rate and a good taste.

Tabletting step:

The components listed in Table 6c below were homogeneously mixed.

The homogeneous obtained mixture was introduced in a tabletting machine, with a tabletting speed of 60000 tablets/hour. The punches used were 12 mm polo punches. The internal wall of the punches were pulverized with magnesium stearate and then 400.00 mg of the mixture were introduced in each punch.

TABLE 6c

| Components | Unit composition (mg) |
|---|---|
| terbinafine HCl coated granules | 230.16* |
| Pearlitol 400 DC | 104.84 |
| Kollidon CL | 25.00 |
| AcDisol | 20.00 |
| Aspartam | 8.00 |
| Tutti Frutti flavor | 9.00 |
| Syloïd FP 244 | 3.00 |
| Magnesium stearate | qsp lubricating |
| Total weight of tablets | 400.00 |

*corresponds to 125 mg terbinafine base.

The hardness of the tablets was 39N

Thickness: 4.52 mm.

Disintegration time: less than 30s.

The taste was pleasant.

### Example 7 [99026/0058]

The granules obtained in example 6 above (granulation step) were coated at a coating amount of 25% and of 30%

The respective coating compositions are given in table 7b below:

TABLE 7b

| | Coating ratio 25% | | Coating ratio 30% | |
|---|---|---|---|---|
| Components | Amount (g) | % | Amount (g) | % |
| Granules of | 8263.0 | 74.07 | 8201.0 | 94.64 |
| terbinafine HCl | | | | |
| Eudragit ® L100-55 | 1446.0 | 12.96 | 232.5 | 2.68 |
| Eudragit ® RL100 | 619.7 | 5.56 | 99.6 | 1.15 |
| Ethyl phthalate | 206.6 | 1.85 | 33.2 | 0.38 |
| Syloïd 244 FP | 619.7 | 5.56 | 99.6 | 1.15 |
| Ethanol USP/PE | 14460 | | 2325 | |

Said coated granules present a good dissolution rate and a good taste.

Tabletting Step:

The components listed in Table 7c below were homogeneously mixed.

The homogeneous obtained mixture was introduced in a tabletting machine, with a tabletting speed of 50000 tablets/hour. The punches used were 12 mm flat punches with bevelled edges. The internal wall of the punches were pulverized with magnesium stearate and then 400.00 mg of the mixture were introduced in each punch.

TABLE 7c

| | Coating | |
|---|---|---|
| | amount of 25% | Coating amount of 30% |
| Components | Unit composition (mg) | Unit composition (mg) |
| terbinafine HCl coated | 200.39* | 210.78* |
| granules | | |
| Pearlitol 400 DC | 134.61 | 124.22 |
| Kollidon CL | 25.00 | 25.00 |
| AcDisol | 20.00 | 20.00 |
| Aspartam | 8.00 | 8.00 |
| Tutti Frutti flavor | 9.00 | 9.00 |
| Syloïd FP 244 | 3.00 | 3.00 |
| Magnesium stearate | qsp lubricating | qsp lubricating |
| Total weight of tablets | 400.00 | 400.00 |

*correspond to 125 mg terbinafine base.

The hardness of the tablets was respectively 36N and 44N.

Thickness was: 4.30 mm in each case.

Disintegration time: less than 30s.

The taste was pleasant

### Exemple 8

The granules obtained in example 6 above (granulation step) were coated at a coating amount of 15% and of 10%

The respective coating compositions are given in table 8b below:

TABLE 8b

| | Coating amount | | Coating amount | |
|---|---|---|---|---|
| | of 15% | | of 10% | |
| Components | Amount (g) | % | Amount (g) | % |

| | | | | |
|---|---|---|---|---|
| terbinafine granules | 6572.0 | 93.87 | 8000.0 | 87.62 |
| Eudragit ® L100-55 | 214.7 | 3.07 | 560.0 | 6.14 |
| Eudragit ® RL100 | 92.0 | 1.31 | 240.0 | 2.63 |
| Ethyl phthalate | 30.7 | 0.44 | 80.0 | 0.88 |
| Syloid 244 FP | 92.0 | 1.31 | 240.0 | 2.63 |
| Ethanol USP/PE | 2146.7 | | 5600 | |

Said coated granules present a dissolution rate of more than 80% after 30 mn.

Tabletting Step:

The components listed in Table 8c below were homogeneously mixed.

The homogeneous obtained mixture was introduced in a tabletting machine, with a tabletting speed of 50000 tablets/hour. The punches used were 12 mm flat punches with bevelled edges. The internal wall of the punches were pulverized with magnesium stearate and then 400.00 mg of the mixture were introduced in each punch.

TABLE 8c

| | Coating ratio of 15% | Coating ratio of 10% |
|---|---|---|
| Components | Unit composition (mg) | Unit composition (mg) |
| terbinafine coated | 180.16* | 169.12* |
| granules | | |
| Pearlitol 400 DC | 154.84 | 165.88 |
| Kollidon CL | 25.00 | 25.00 |
| AcDisol | 20.00 | 20.00 |
| Aspartam | 8.00 | 8.00 |
| Tutti Frutti flavor | 9.00 | 9.00 |
| Syloïd FP 244 | 3.00 | 3.00 |
| Magnesium stearate | qsp lubricating | qsp lubricating |
| Total weight of tablets | 400.00 | 400.00 |

*correspond to 125 mg terbinafine base.

The hardness of the tablets was respectively 35 and 38 N

Disintegration time: less than 30s.

The taste was pleasant

1. Granules of allylamine- or benzylamine-anti-mycotic, or one of its pharmaceutically acceptable salts, wherein the granules are coated and contain:
microcrystals of an allylamine- or benzylamine-anti-mycotic, or one of its pharmaceutically acceptable salts,
at least one binder selected from the group consisting of cellulosic polymers, such as ethylcellulose, hydroxypropylcellulose and hydroxypropylmethylcellulose, acrylic polymers such as insoluble acrylate ammoniomethacrylate copolymer, polyacrylate or polymethacrylic copolymer; povidones, polyvinylalcohols and mixtures thereof,
optionally a diluent agent selected from the group comprising cellulosic derivatives, microcrystalline cellulose, starches, lactose, polyols such as mannitol and/or an antistatic agent selected from the group comprising micronised or non micronised talc, and/or a permeabilising agent selected from the group cosisting of colloidal silica and precipitated silica.

2. Granules according to claim 1, wherein the allylamine- or benzylamine-anti-mycotic is selected from the group consisting of butenafine, naftifine, and preferably terbinafine.

3. Granules according to claim 1 presenting a granulometric repartition such that at least 50%, preferably at least 70%, of the granules have a particle size ranging between 125 and 355 μm and less than 15% of the granules have a particle size less than 90 μm.

4. Granules according to claim 1 comprising:
from 10% to 80% of granules of the allylamine- or benzylamine-anti-mycotic, or one of its pharmaceutically acceptable salts,
at most 15%, preferably at most 5% by weight of the binder, relative to the weight of allylamine- or benzylamine-anti-mycotic, or one of its pharmaceutically acceptable salts,
at most 2%, preferably 5% by weight of the antistatic agent, relative to the weight of the granules of the allylamine- or benzylamine-anti-mycotic, or one of its pharmaceutically acceptable salts.

5. Granules according to claim 1, wherein they are coated with a coating composition containing at least one coating polymer selected from the group consisting of cellulosic polymers such as ethylcellulose, hydroxypropylcellulose and hydroxypropylmethylcellulose, acrylic polymers such as insoluble acrylate ammonio-methacrylate copolymer, polyacrylate, or methacrylic copolymers used, alone, in combination or in admixture with pH-dependent polymers, and their mixtures, and optionally a permeabilising agent, plasticizers, and soluble agents such as polyols.

6. Granules according to claim 1, comprising:
from 10% to 95% of granules of allylamine- or benzylamine-anti-mycotic, or one of its pharmaceutically acceptable salts, preferably terbinafine HCl,
from 5 to 60%, preferably from 5 to 40% and even more preferably from 10 to 20% of a coating polymer, the percentages being expressed by weight relative to the weight of the granules of allylamine- or benzylamine-anti-mycotic, or one of its pharmaceutically acceptable salts,
from 0 to 30%, preferably 10% by weight of plasticizers, preferably ethyl phthalate, the percentages being expressed by weight relative to the weight of the amount of the coating polymer,
from 0 to 10% of a permeabilising agent, preferably colloidal silica, the percentages being expressed by weight relative to the weight of the amount of the coating polymer, and
from 0 to 10% of an antistatic agent, preferably micronised talc the percentages being expressed by weight relative to the weight of the amount of the coating polymer.

7. Granules according to claim 1, further comprising an overcoating essentially consisting in acrylic polymer, cellulosic polymer, thermoplastic polymer or mixtures thereof.

8. Process for the preparation of granules according to claim 1, comprising the successive steps consisting in:
dry mixing the microcrystals of the allylamine-or benzylamine-anti-mycotic or one of its pharmaceutically active salts optionally with an antistatic agent and/or a diluent agent, and/or a permeabilising agent;
granulating the mixture obtained in the above step by spraying of a solution or suspension of the binder,
coating the thus obtained granules with a suspension of the coating composition,
optionally, applying onto the thus obtained coated granules an overcoating based on acrylic polymer, cellulosic polymer, thermoplastic polymer or mixtures thereof,
drying the thus obtained coated granules.

9. Process according to claim 8, wherein the granulation is a bottom spray granulation, tangential spray granulation, top spray granulation or high shear granulation, preferably bottom spray granulation.

10. Orodispersible tablets comprising coated granules according to claim 1 or prepared according to claim 8, and a mixture of excipients comprising at least one disintegrating agent, a soluble diluent agent, a lubricant and optionally a swelling agent, a permeabilising agent, sweeteners, flavorings and colors.

11. Orodispersible tablets according to claim 10, in which the ratio of the mixture of excipients to the coated granules is 0.4 to 10, preferably 0.4 to 5 and even more preferably 1 to 2 parts by weight, the mixture of excipients comprising:
at least one disintegrating agent,
a soluble diluent agent which presents binding properties,
a lubricant,
a permeabilising agent,
and optionally sweeteners, flavorings and colors.

12. Orodispersible tablets according to claim 10, in which the disintegrating agent is selected from the group consisting of croscarmellose, crospovidone and mixtures thereof.

13. Orodispersible tablets according to claim 10 in which the soluble diluent agent with binding properties consists of a polyol having less than 13 carbon atoms and being either in the form of the directly compressible product with an average particle size of 100 to 500 μm, or in the form of a powder with an average particle size of less than 100 μm, this polyol preferably being selected from the group comprising mannitol, xylitol, sorbitol and maltitol, it being understood that sorbitol cannot be used alone and that, in the case where there is only one soluble diluent agent with binding properties, it is used in the form of the directly compressible product, whereas in the case where there are at least two soluble diluent agents with binding properties, one is present in the directly compressible form and the other is present in powder form, it then being possible for the polyols to be the same, the ratio of directly compressible polyol to powder polyol being 99/1 to 20/80, preferably 80/20 to 20/80.

14. Orodispersible tablets according to claim 10, in which the permeabilizing agent is selected from the group comprising especially silicas with a high affinity for aqueous solvents, such as the precipitated silica better known by the trade mark Syloïd®, maltodextrins, β-cyclodextrins and mixtures thereof.

15. Orodispersible tablets according to claim 10, in which the lubricant is selected from the group consisting of magnesium stearate, stearic acid, sodium stearyl fumarate, micronised polyoxyethyleneglycol (micronised Macrogol 6000), leukine, sodium benzoate and mixtures thereof.

16. Orodispersible tablets according to claim 10, in which the proportion of disintegrating agent is 1 to 20% by weight, preferably 5 to 15% by weight, and the proportion of soluble agent is 30 to 90% by weight, preferably 30 to 50% by weight, based in each case on the weight of the tablet.

17. Method of treatment of a condition selected from the group comprising onychomycosis caused by dermatophyte fungi, tinea capitis, fungal infections of the skin, tinea corporis, tinea cruris, tinea pedis, and yeast infections of the skin caused by the genus Candida, e.g. Candida albicans, systemic mycosis, mycosis by azole-resistant strains, e.g. in combination with a 14-alpha-methyldimethylase inhibitor, or infections with Helicobacter pylori, by administering to patients, in particular to children by oral route orodispersible tablets according to claim 10.
